(19) United States
(12) Patent Application Publication
(10) Pub. No.: US 2014/0088461 A1
(43) Pub. Date: Mar. 27, 2014

(54) HEALTH MONITOR

(71) Applicant: X2 Biosystems, Inc., Seattle, WA (US)

(72) Inventors: Christoph Mack, Seattle, WA (US); Richard T. Able, Tacoma, WA (US)

(73) Assignee: X2 Biosystems, Inc., Seattle, WA (US)

(21) Appl. No.: 14/040,157

(22) Filed: Sep. 27, 2013

Related U.S. Application Data
(60) Provisional application No. 61/706,532, filed on Sep. 27, 2012.

(51) Int. Cl. A61B 5/00; A61B 5/11

(52) U.S. Cl. CPC A61B 5/6804; A61B 5/1107
USPC 600/595

(57) ABSTRACT

A health monitoring device includes a sensor configured to be worn by an individual on a portion of the lower leg such as a foot, ankle, or a portion of the leg just above the ankle. The health monitoring device is configured to track expansion of the body appendage over time, thereby allowing a healthcare professional to see changes in the diameter that may be caused by the pooling of body fluids.

### PRIORITY CLAIM

This application claims the benefit of U.S. Provisional Application Ser. No. 61/706,532 filed Sep. 27, 2012, the contents of which are hereby incorporated by reference.

### BACKGROUND OF THE INVENTION

There are several health conditions that are associated with the pooling of blood in the body, particularly in the area of the lower legs, ankles, or feet. For example, individuals with COPD sometimes experience such swelling. In such cases, pulmonary hypertension may be a complication that occurs as a result of an increase in pressure in the blood vessels of the lungs. The increased pressure damages the vessels and causes blood to back up in the veins within the body. Excess fluid leaks into the surrounding tissues, and the force of gravity causes the fluid to pool in lower extremities such as the legs, ankles, and feet. Other conditions can also be indicated by such swelling. As another example, congestive heart failure may result in a reduced amount of blood in the kidneys, thereby causing fluid and water retention which results in swollen ankles, legs, or feet.

In many cases, early awareness of the symptomatic swelling may improve the ability to diagnose and treat the health condition. Similarly, close monitoring of swelling in extremities may allow healthcare professionals to determine when the symptoms have worsened, thereby allowing treatment at the earliest opportunity.

Presently, however, there is no ability to track such swelling except to the extent that it is noticed by the affected individual or by a healthcare professional during an examination. In many cases, swelling or pooling of fluids may be at its peak when the individual is sleeping or otherwise unaware of the condition Likewise, the individual may be aware of some amount of swelling but unable to detect changes in the magnitude. Accordingly, there is no current ability to carefully monitor this important symptom associated with significant health issues.

### SUMMARY OF THE INVENTION

A preferred version of the invention includes at least one band configured to be worn by an individual around a portion of the lower leg such as a foot, ankle, or a portion of the leg just above the ankle. The health monitoring device is configured to track the diameter of the body appendage over time, thereby allowing a healthcare professional to see changes in the diameter that may be caused by the pooling of body fluids. Further investigation by the healthcare professional may facilitate treatment of the cause of the symptom at an earlier time than might have been otherwise possible.

In some versions of the invention, data associated with the tracked parameter such as the circumference of the appendage is stored with the health monitor for later access and review.

In other versions of the invention, data is transferred contemporaneously in a wired or wireless fashion to a remote device which allows for local analysis and evaluation of a health condition.

In some versions, the data is transferred to a remote device such as that of a health care professional for remote, rather than local, analysis.

### BRIEF DESCRIPTION OF THE DRAWINGS

Preferred and alternative examples of the present invention are described in detail below with reference to the following drawings.

FIG. 1 is an illustration of an article of clothing, preferably a sock, configured to be worn by a user and containing sensing components.

FIG. 2 is an illustration of a preferred sensing band that may be incorporated into a device such as the sock of FIG. 1 or may be configured as a separate band.

FIG. 3 is an illustration of an alternate preferred sensing band.

FIG. 4 is a block diagram illustrating components for a preferred health monitor.

FIG. 5 is an illustration of a preferred health monitor system including a user computer and a remote health care computer.

FIG. 6A is a top view of a preferred sensing band.

FIG. 6B is a top view of a preferred sensing band, shown in an unfastened position.

FIG. 7 is a graph of a representative collection of sensed data corresponding to a diameter of a wearer's appendage such as a lower leg.

### DETAILED DESCRIPTION OF THE PREFERRED EMBODIMENT

In one example of the invention, a health monitoring device includes at least one band configured to be worn by an individual around a portion of the lower leg such as a foot, ankle, or a portion of the leg just above the ankle. The health monitoring device is configured to track the diameter, circumference, or other parameter of expansion or contraction associated with swelling of the body appendage over time, thereby allowing a healthcare professional to see changes that may be caused by the pooling of body fluids. Further investigation by the healthcare professional may facilitate treatment of the cause of the symptom at an earlier time than might have been otherwise possible.

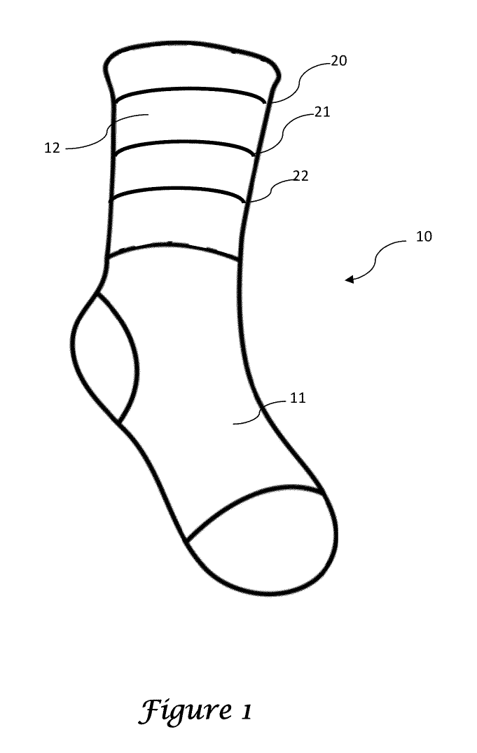

With reference to FIG. 1, in one example of the invention a sock or other such foot covering 10 is provided to be worn by an individual. The sock includes a foot covering portion 11 and an ankle and/or lower leg covering portion 12, with one or more bands 20, 21, 22 positioned adjacent one another and integrated into portions of the sock that encircle the lower leg and ankle of the wearer. While three bands are illustrated, any number of bands may be used in accordance with the invention. As discussed further below, each of the bands are created in a manner that allows the device to monitor the diameter of the portion of the body inserted into the band. Accordingly, where three such bands are provided as illustrated in FIG. 1, the device will monitor the diameter of the foot or leg of the wearer in three separate locations.

In other versions of the invention, the one or more sensing bands may be positioned to encircle the ankle or a portion of the foot of the wearer, rather than the lower leg as illustrated. In some versions, the bands may be located anywhere along the sock or foot covering, including about the ankle or about desired locations encircling the foot covering portion 11.

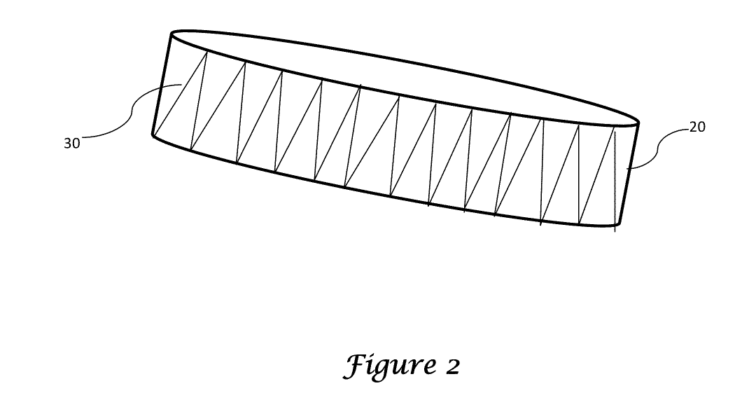

With reference to FIG. 2, each band 20 preferably includes a fine wire 30 sewn into an elastic upper portion of the sock. The wire is configured as a strain gauge, thereby allowing the gauge to detect both expansion and compression along a perimeter formed by the band, as well as expansion or compression in a perpendicular direction, along a vertical axis that is orthogonal to the plane defining the diameter of the band. Accordingly, the strain gauge will detect an increase in the diameter of the band (and therefore the circumference of the band), thereby indicative of swelling or a pooling of fluids in the vicinity of the band. Most preferably, the wire 30 will be incorporated into the sock or band in a fashion that allows for the determination of an actual circumference of the wire, and therefore the appendage encircled by the wire. At the time of manufacture, the wire is preferably calibrated such that a particular electrical conductance (or, alternately stated, impedance) of the wire is associated with a particular diameter or circumference of the sock or band into which the wire is woven or otherwise attached. Thereafter, changes in the impedance of the wire allow the calculation of the deformation, and therefore the diameter or circumference, of the loop formed by the wire.

In addition, the strain gauge will detect vertical sag or compression that may be associated with a vertical movement of the bands such as when the sock slides up or down on the leg of the wearer. Although illustrated in FIG. 2 as comprising a pattern of alternating wires extending diametrically about the upper portion of sock, additional wires may be woven into the material in a pattern configured to detect vertical tension or compression indicative of movement of the sock in a vertical fashion. As the sock sags it will fold over itself at least somewhat or the material will compress in various portions, thereby creating a different impedance than would be the case if it is pulled up into a desired position. In addition, FIG. 2 notionally shows a strain gauge in the form of a wire encircling the sock, while in other embodiments it need not fully encircle the sock. Rather, the strain gauge may be formed along a much smaller portion of the sock sufficient to detect a change in the diameter, length, or other configuration of the sock. In such a version, a localized expansion of the sensor or strain gauge would be presumed to reflect an increase in the diameter of the appendage at that location.

As discussed above and illustrated with respect FIG. 1, one version of the invention incorporates a sensor configured to detect expansion and contraction within a sock. In other versions of the invention, the sensor may be incorporated into a band that is not a part of a sock. Thus, as seen in FIG. 2 the band 20 may stand alone and have incorporated sensors without being part of a sock. Alternatively, the same band as illustrated in FIG. 2 may be integrated into a sock or other article of clothing.

In yet other versions of the invention, the sensor or strain gauge may be directly adhered to the skin of the individual. In such a version of the invention, the sensor may be attached to a substrate such as a sticker that may be directly attached to the skin of the wearer. In the same fashion as described above, the strain gauge would be configured to detect expansion or compression of the strain gauge, thereby indicating pooling or swelling in the location where the sensor is attached. Most preferably, the sticker would include more than one strain gauge or sensor for redundancy and to detect changes in multiple directions. Likewise, the sticker is preferably formed from a material such as gauze that has some natural ability to expand and contract such that it will expand and contract with the skin to which it is adhered. In alternate versions, the sticker may be formed from a thin layer of an elastomeric material such as a gel or thin silicone layer that is able to expand and contract with the skin.

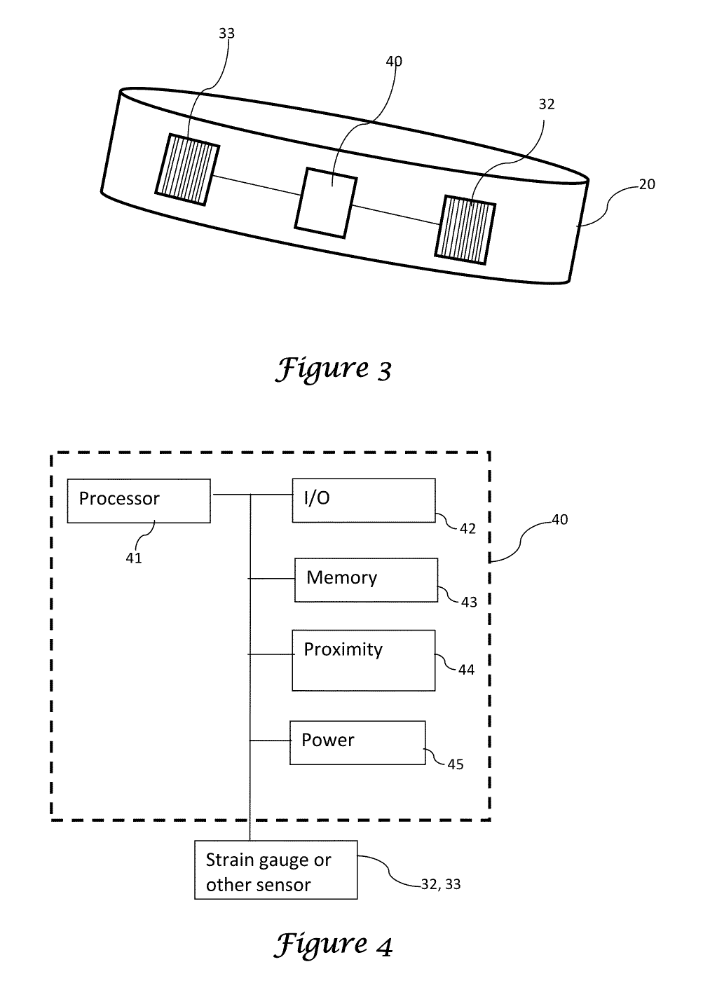

The monitor may include onboard components to collect, store, and transmit data from the sensors for evaluation later or in real time. In one example, as seen in FIG. 3, the band may include two or more strain gauges (or other sensors) 32, 33 in communication with electrical components 40 for collecting the sensor data.

With reference to FIG. 4, the electrical components 40 of the monitor may include a processor 41 and a memory 43 in communication with the processor. The memory preferably includes stored programming instructions operable by the processor to allow the processor to collect data from the sensors and to store it in the memory. In one version, one or more additional sensors such as a proximity sensor or other sensor 44 is provided to determine when the monitor is in place and worn by the user. The proximity or other such sensor is likewise in signal communication with the processor and is preferably associated with the strain gauge 32, 33 or other sensor data to confirm that the sensor data is valid because the monitor is in use. A power supply such as a small button battery is also provided. It should be appreciated that while each of the above components is shown in FIGS. 3 and 4 as being positioned on a band, they need not fully encircle a leg or ankle of the wearer, and instead may be incorporated into a sticker or otherwise attached directly to the skin of the wearer as described above.

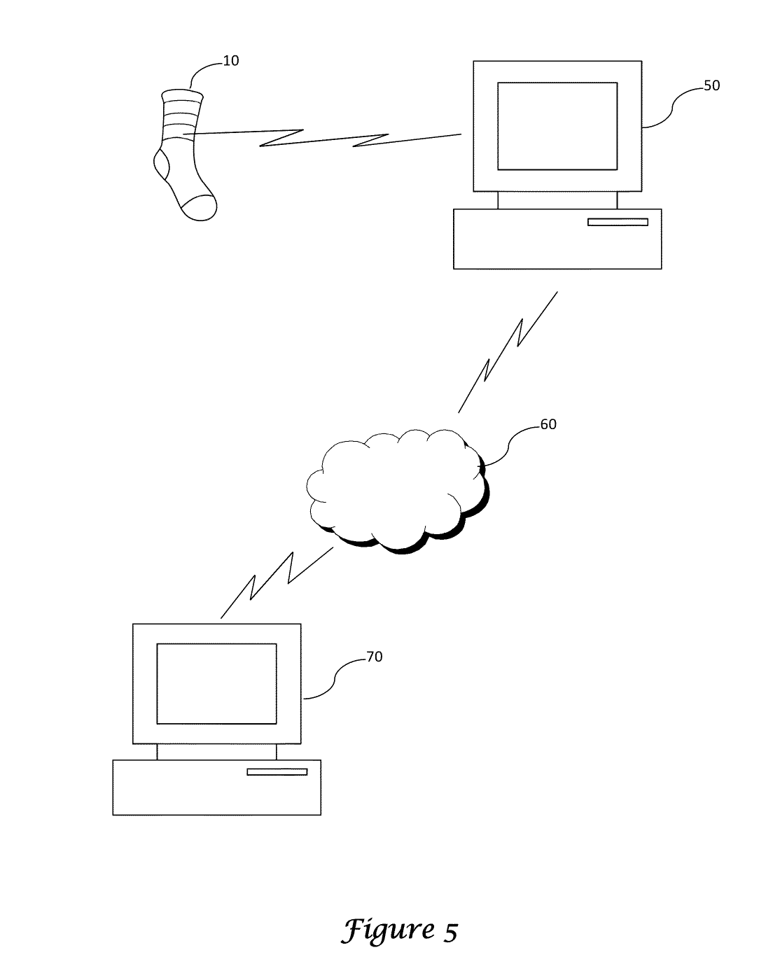

In a preferred example of the invention, the monitor is configured to allow data gathered by the monitor to be transferred to a computer for subsequent evaluation (or, alternatively, for contemporaneous real time evaluation). Thus, in one configuration as illustrated in FIG. 5, an input/output component 42 (see FIG. 4) on the monitor 10 (including the version formed as a sticker or a separate band 20, as illustrated in FIG. 3) includes a wireless transmitter (such as Wi-Fi, Bluetooth, or the like) allowing the data to be wirelessly transferred to a local computer 50. The local computer may further include stored programming instructions allowing for the collection and evaluation of the sensor data locally. In some versions, the local computer may be configured to store the collected data over time, further calculating average data (such as average leg or ankle diameter or circumference data at particular locations along the leg or ankle), thereby allowing the local computer to compare data for a particular day or time with a baseline average value.

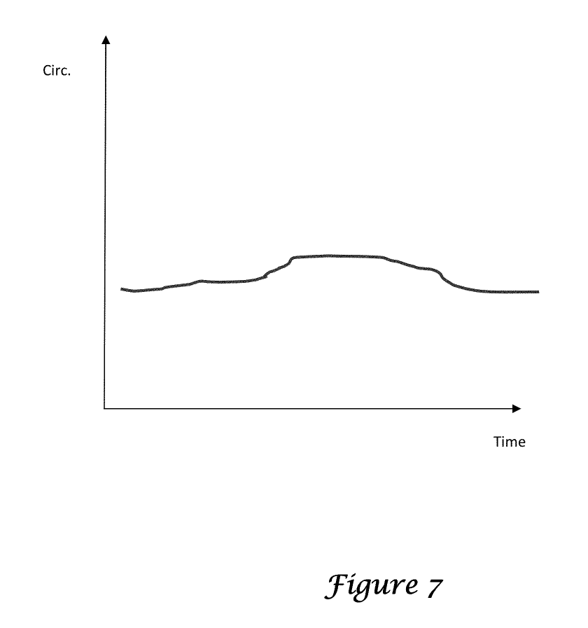

The local computer may likewise be in communication with a remote computer 70, directly or over a network 60 such as the Internet. In some versions the remote computer 70 may be that of a doctor or other healthcare professional. The remote computer may be configured to evaluate the data in the same fashion as identified above, or may further include similar data aggregated from a multitude of patients in order to allow comparison of individual data or trends from a single-user with aggregated data or trends from a larger population. The circumference of wearer's leg or ankle may be presented on the local or remote computer as a function of time, for example as illustrated in FIG. 7.

If desired, a graphic representation of the circumference (or other indicator of expansion) may be presented along with baseline or average data for the user Likewise, if multiple bands are incorporated into a sock or otherwise worn by the user, the graph may illustrate each of the multiple bands at the same time, thereby allowing a simultaneous representation that might indicate expansion or pooling at one location but not in another.

In some versions of the invention, the local computer 50, remote computer 70, or both may include threshold values stored in memory and programming instructions causing the computer to trigger an alarm if certain criteria are met. For example, the local computer may include a value for a diameter or circumference of the leg or ankle of the wearer (or other parameter indicative of swelling) at a particular location, and a further continually compare actual measured values from the sensors with the stored threshold value in real time. If the actual values exceed the threshold, the programming instructions may cause the computer to sound an alarm such as a tone, bell, or other audible or visual signal.

While an actual circumference or diameter may be calculated for a particular appendage as desired, in some versions the invention need not make such actual determinations. Instead, the system may evaluate the attached sensors or gauges for changes from initial positions. For example, in the version in which the sensor is applied as a sticker, the system (such as the local computer 50) may evaluate changes in impedance in order to determine changes in physiology. The computer memory may further contain one or more threshold values representative of acceptable or unacceptable degrees of change from the initial determined impedance value to allow the system to determine degrees of variation and to sound an alarm or otherwise record such changes as being associated with an unhealthy condition. Similarly, the vertical axis labeled “Circ.” indicating “circumference” in FIG. 7 may alternatively be understood to indicate an increase in the magnitude of sensor readings such that expansion detected by the sensor is indicated by movement upward along the vertical axis.

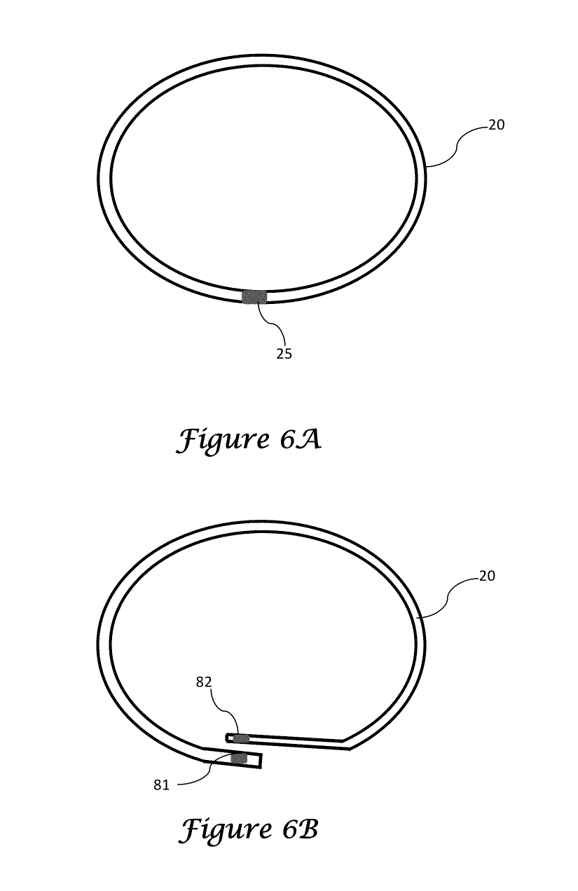

As described above, the sensors may be incorporated into a band or woven or otherwise attached to a sock in some versions of the invention. Thus, in some examples the monitor is formed as a loop, allowing the foot and leg to be inserted into the loop. In other versions, the monitor may be formed as a length of material that may be fastened together to form a loop. As shown in FIG. 6B, a pair of mating fasteners 81, 82 such as a snap, Velcro, button, or other such means for joining two ends of the loop together may be used.

The monitor may also include a position sensor 25 as shown in FIG. 6A configured to determine whether the monitor is in place. In one example, the position sensor may be an optical sensor to detect received light at the location of the sensor. The sensor is directed inward, to detect light received from inside the diameter of the band 20. The sensor would be expected to receive essentially no light when the band is in place, positioned about the leg or ankle of the user. In such a case, the processor would conclude that the band is being worn by the user. Other forms of position sensors may also be used, such as a temperature sensor, capacitive sensor, a pair of electrodes, or other devices. In each case the position sensor is in signal communication with the processor so that the memory can store data from the position sensor. In one preferred version of the invention, the monitor stores a reading from the position sensor (such as capacitance or an amount of light) at a time when the device is first positioned in place, and thereafter compares the initial reading with subsequent detected readings. If there is variation below a desired level, the monitor may determine that the monitor has fallen down or has been removed. In one version, the monitor may sound an alarm if such a condition is determined.

In the above described examples of the invention, a strain gauge is primarily described as the preferred version of a sensor for detecting the diameter or expansion/contraction of the leg or ankle of the wearer. Other forms of sensors may also be used such as piezoelectric sensors, optical sensors, magnetic sensors, capacitive sensors, or yet others.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A health monitor, comprising:
a first sensor configured to detect an expansion or contraction of a portion of an appendage of a user wearing the health monitor;
a processor in communication with the first sensor;
a memory containing stored programming instructions, the programming instructions operable by the processor to cause the memory to store data received from the first sensor.

2. The health monitor of claim 1, wherein the first sensor is formed as a band configured to encircle the appendage.

3. The health monitor of claim 1, wherein the first sensor is incorporated into a sock.

4. The health monitor of claim 3, further comprising a second sensor configured to detect an expansion or contraction of a separate portion of the appendage of the user wearing the health monitor.

5. The health monitor of claim 1, wherein the first sensor is attached to a sticker configured to be adhered to a portion of skin of the user.

6. The health monitor of claim 5, wherein the sticker comprises an adhesive applied to a gauze material.

7. The health monitor of claim 1, wherein the first sensor is incorporated into a sock.

8. The health monitor of claim 1, further comprising an input/output component, the input/output component being configured to communicate with a local computer.

9. The health monitor of claim 8, wherein the input/output component is configured to transfer the data from the first sensor to the local computer.

10. The health monitor of claim 9, wherein the local computer includes a component configured to compare the data transferred from the first sensor with a threshold value, the threshold value being associated with a degree of expansion or contraction experienced by the first sensor.

11. The health monitor of claim 10, wherein the local computer is further configured to trigger an alarm when the data from the first sensor is outside the threshold value.

12. The health monitor of claim 9, wherein the local computer is configured to transfer the data or a representation of the data from the local computer to a remote computer over a network.

13. The health monitor of claim 12, wherein the remote computer includes a component configured to compare the data transferred from the first sensor with a threshold value, the threshold value being associated with a degree of expansion or contraction experienced by the first sensor.

14. The health monitor of claim 1, wherein the stored programming instructions are configured to compare the data from the first sensor with a threshold value, the threshold value being associated with a degree of expansion or contraction experienced by the first sensor, the programming instructions further being configured to trigger an alarm when the data from the first sensor is outside the threshold value.

15. The health monitor of claim 1, further comprising a proximity sensor configured to detect proximity of the health monitor with a user.

16. A health monitor, comprising:
a sensor, the sensor being attached to a substrate material, the substrate material being configured to allow for expansion and contraction whereby the sensor is configured to detect an expansion or contraction of the substrate material, the sensor and the substrate material further being sized and configured to be attached to an appendage of a person, whereby expansion or contraction of the appendage of the person causes corresponding expansion or contraction of the substrate material when the substrate material is attached to the appendage;
a processor carried by the substrate material and in communication with the sensor;
a memory carried by the substrate material and containing stored programming instructions, the programming instructions operable by the processor to cause the memory to store data received from the sensor.

17. The health monitor of claim 16, wherein the sensor comprises a plurality of sensors, the plurality of sensors being spaced apart from one another on the substrate material.

18. The health monitor of claim 16, further comprising an input/output component carried by the substrate, the input/output component being configured to communicate with computer to receive and store the data received from the sensor.

19. The health monitor of claim 18, wherein the computer further comprises a processor and a memory, the memory containing stored programming instructions to track changes in expansion of the appendage over time.

20. A method for monitoring health of a user, comprising:
providing a health monitor in accordance with claim 16;
attaching the health monitor to the person; and
determining whether the appendage of the person has expanded or contracted beyond a threshold value.
